(19) United States
(12) Patent Application Publication
(10) Pub. No.: US 2022/0088307 A1
(43) Pub. Date: Mar. 24, 2022

(54) PORTABLE AUTO INJECTION APPARATUS WITH SHORT LENGTH

(71) Applicant: GFYHEALTH INC., Seongnam-si, Gyeonggi-do (KR)

(72) Inventor: Tae Hyun LEE, Seongnam-si, Gyeonggi-do (KR)

(73) Assignee: GFYHEALTH INC., Seongnam-si, Gyeonggi-do (KR)

(21) Appl. No.: 17/257,644

(22) PCT Filed: Dec. 11, 2020
(86) PCT No.: PCT/KR2020/018137

§ 371 Date: Jan. 4, 2021

(30) Foreign Application Priority Data
Dec. 27, 2019 (KR) 10-2019-0176004

(51) Int. Cl. A61M 5/20; A61M 5/142; A61M 5/315

(52) U.S. Cl. CPC A61M 5/20; A61M 5/14244; A61M 2005/3125; A61M 2005/3152; A61M 5/31576

(57) ABSTRACT

A portable auto injection apparatus with short length is provided. A portable auto injection apparatus with short length according to an embodiment of the present invention includes a syringe to store an injection solution; a piston coupled to one end of the syringe and driving to expand/contract to discharge the injection solution to an outlet formed at the other end of the syringe; a push member coupled to one end of the piston and driving the piston; and a driving unit coupled to the push member and driving the push member.

### TECHNICAL FIELD

The present invention relates to a portable auto injection apparatus with short length.

### BACKGROUND

Diabetes is a disease that occurs when the body cannot make enough insulin or use insulin properly and cannot convert blood sugar, or glucose, into energy.

Currently, diabetes treatment methods are broadly classified into diet, exercise therapy, drug therapy, and insulin injection, and pancreatic transplantation is also being attempted.

There are several methods of insulin injection, among which there is a method using an insulin pump.

The insulin pump is a device developed to relieve the inconvenience of having to get an insulin injection every day.

The insulin pump can automatically and continuously administer insulin, and also adjust the time and dose of insulin supply as desired.

Since this method of using the insulin pump is a method that can make the change in blood insulin concentration obtained by insulin injection most similar to the change in blood insulin concentration due to the insulin secretion reaction of a normal person among the insulin injection methods that have been introduced so far, the number of patients using insulin pumps is rapidly increasing in developed countries.

The insulin pump is generally referred to as a long-time injection auto-injector, insulin syringe, insulin auto-injector, and portable auto injection apparatus, etc., and has a structure in which a push means for pushing a syringe piston is coupled to a housing accommodating an injection syringe. This can be exemplified by Japanese Utility Model Laid-Open No. Showa 52-3292 and U.S. Pat. No. 4,417,889.

In addition, taking Korean Patent Registration No. 10-0353229 developed in Korea as an example of related art, it is a portable auto-injector that allows a rotating shaft and a push plate to be combined with a housing in the correct position after separating the rotating shaft from the housing when refilling a syringe with injection solution after use, and as shown in FIG. 1, a syringe using a conduit 21 connected to a connecting hole 22 is connected through a cover 33, and the cover 33 is coupled to an upper end of the housing 37 so as to be sealable. The housing 37 corresponding to the cover 33 contains a syringe 23, a piston 24, a piston pushing means 25, and a rotating shaft 26 that provides power through a power supply means 40. The housing 37 contains a control button unit 36 for commanding a control board (not shown), a display 38 such as an LCD that displays a control state, a battery cover 35 for fixing a battery providing power, and a reset button 34 that performs a reset function. However, such a conventional insulin pump has a structure that a syringe for accommodating insulin, a piston for pushing and supplying insulin from the syringe, and a rotation shaft for providing forward and retreat power of the piston are vertically arranged.

That is, of course, since the syringe, the piston and the rotating shaft must be accommodated in a vertical state in the housing, the length of the housing must inevitably be long enough to accommodate it, and the length of the housing cannot be lowered below this height.

Therefore, in the case of the conventional insulin pump, since it is impossible to shorten the overall length, there is a problem that it is not only inconvenient to carry due to its large size, but also has many restrictions when forming its shape.

### SUMMARY

The present invention is directed to providing a portable auto injection apparatus with short length capable of shortening the length of a portable auto injection apparatus.

A portable auto injection apparatus with short length according to an embodiment of the present invention includes a syringe to store an injection solution;

a piston coupled to one end of the syringe and driving to expand/contract to discharge the injection solution to an outlet formed at the other end of the syringe; a push member coupled to one end of the piston and driving the piston; and a driving unit coupled to the push member and driving the push member.

In addition, the push member may be a flexible push member having an elastic restoring force.

In addition, the piston may include a head which is in contact with the inner surface of the syringe and moves in a sealed state, and a body in which one end is coupled to the head and the other end is fixedly coupled to one end of the syringe.

In addition, the body may include at least two or more rod members coupled to each other in a multistage structure, and the rod member may expand and contract according to the driving of the push member.

In addition, the push member may include a rack gear formed on a side of the push member and formed in a saw tooth shape at a predetermined interval, and the rack gear may be coupled to the driving unit to drive.

In addition, the push member may further include a scale formed on one side of the rack gear for checking an amount of the injection.

In addition, the portable auto injection apparatus with short length may further include a power providing member to generate power transmitted to the driving unit; a sensor to recognize the scale; and a controller to control the power providing member by receiving information from the sensor.

In addition, the portable auto injection apparatus with short length may further include a display to output a value recognized by the sensor.

In addition, the portable auto injection apparatus with short length may further include a support for supporting one side of the push member to guide a moving direction of the push member.

In addition, the push member may be formed of a semi-curved wire rod.

In addition to the technical problems of the present invention mentioned above, other features and advantages of the present invention will be described below or will be clearly understood by those of ordinary skill in the art from such techniques and descriptions.

According to the present invention there are following effects.

The present invention may shorten the length of the piston by forming the piston in a structure that expands and contracts.

The present invention may shorten the length of a space required for the push member by forming the push member as a flexible push member having an elastic restoring force.

The present invention may reduce the size of the portable auto injection apparatus by shortening the length of the portable auto injection apparatus.

The present invention may reduce cost by configuring a conventional mass-produced syringe to be used as it is.

The present invention may precisely control the amount of the injection solution to be injected by driving the push member including the rack gear formed at a predetermined interval on the push member.

In the present invention a user may easily check the amount of the injected injection solution by including the scale in the push member.

In addition, other features and advantages of the present invention may be newly recognized through embodiments of the present invention.

20

### BRIEF DESCRIPTION OF THE DRAWINGS

The above and other aspects, features, and advantages of the present disclosure will become more apparent to those of ordinary skill in the art by describing embodiments thereof in detail with reference to the accompanying drawings, in which:

FIG. 1 is a perspective view of an insulin pump according to an embodiment of the related art;

FIG. 2 is a schematic perspective view of a portable auto injection apparatus with short length according to an exemplary embodiment of the present invention;

FIG. 3 is a cross-sectional view of a principal part showing a state before driving of a portable auto injection apparatus with short length according to an exemplary embodiment of the present invention;

FIG. 4 is a cross-sectional view of a principal part showing a state after driving of a portable auto injection apparatus with short length according to an exemplary embodiment of the present invention;

FIG. 5 is a schematic top plan view of a portable auto injection apparatus with short length according to an exemplary embodiment of the present invention;

FIG. 6 is a cross-sectional view taken along line A-A′ of the portable auto injection apparatus with short length shown in FIG. 5.

### DETAILED DESCRIPTION OF THE EMBODIMENTS

In the present specification, in adding reference numerals to elements of each drawing, it should be noted that like reference numerals designate like elements as possible throughout the specification even if they are displayed on different drawings.

Meanwhile, the meaning of terms described in the present specification should be understood as follows.

It should be understood that expressions in the singular include plural expressions unless explicitly defined otherwise in context and the scope of rights should not be limited by these expressions.

It should be understood that the terms “comprise or comprising” or “have or having”, when used in this specification, are not preclude the possibility of the presence or addition of one or more other features, integers, steps, operations, components, parts, or a combination thereof.

The present invention will be described more fully hereinafter with reference to the accompanying drawings, in which exemplary embodiments of the invention are shown.

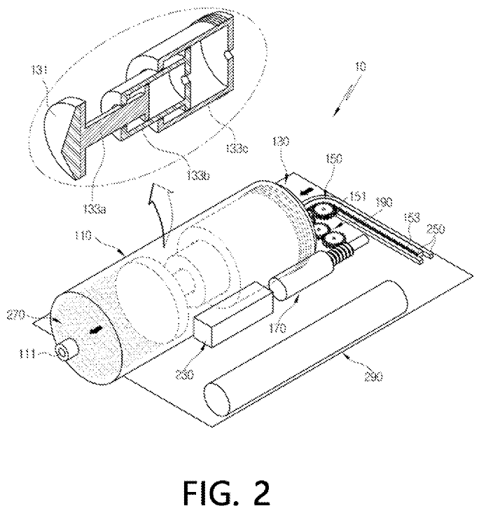

FIG. 2 is a schematic perspective view of a portable auto injection apparatus with short length according to an exemplary embodiment of the present invention.

Referring to FIG. 2, a portable auto injection apparatus with short length 10 according to an embodiment of the present invention includes a piston 130, a push member 150, and a driving unit 190.

The piston 130 is coupled to an inner surface of one end of a syringe 110 to discharge or suck in an injection solution 270 of the syringe 110.

More particularly, the piston 130 has one end fixed to one end of the syringe 110, receives power transmitted to the piston 130, expands and contracts within the syringe 110, and discharges or sucks in the injection solution 270.

In this case, an outer surface of one end of the piston 130 and an inner surface of one end of the syringe 110 may be screwed together in a manner in which the piston 130 is fixedly coupled with the syringe 110.

However, it is not limited thereto, and other methods may be used as long as part of the piston 130 can be fixed when the piston 130 expands and contracts.

For example, one end of the piston 130 may be fitted and fixed between the syringe 110 and a support 250.

In more detail, one end of the piston 130 may form a plate-shaped flange. A longitudinal section of the syringe 110 and one surface of the flange may be in contact with each other, and the other surface of the flange and one surface of the support 250 to be described later may be in contact with each other to be fixed. That is, since the flange of the piston 130 is pinched and fixed between the syringe 110 and the support 250, even if the piston 130 expands and contracts and is driven the coupling is not arbitrarily released. In this case, the component supporting the flange of the piston 130 is not limited to the support 250, and any configuration capable of fixing the flange of the piston 130 may be possible. As an example, it may be possible to add a fixing device capable of separately fixing the flange.

The piston 130 may have a variety of structures in order to expand and contract. In one embodiment, the structure of the piston 130 is configured in a multistage structure, and a more detailed description of the piston 130 will be described later.

The push member 150 provides power for the piston 130 to expand and contract, and may be coupled to one end of the piston 130.

The push member 150 may be used as a handle when the piston 130 coupled with the syringe 110 performs an expansion/contraction movement to suck in the injection solution 270. In this case, the push member 150 and the piston 130 may be coupled so as not to be separated, or the push member 150 and the piston 130 may be integrally configured.

The push member 150 has elastic restoring force and flexibility.

In more detail, since the push member 150 has flexibility, the push member 150 may be bent in a direction guided by the support 250 formed outside. In addition, when not guided by the support 250, the push member 150 may be restored to its original shape by the elastic restoring force of the push member 150.

In this way, the push member 150 may be bent by the support 250 to shorten the length of the portable auto injection apparatus with short length, and the push member 150 out of the bent place may be restored to its original shape by the elastic restoring force and may precisely drive the piston 130 by receiving power from the driving unit 190.

The push member 150 may be made of nylon, which is a polymer compound having flexibility and high elastic restoring force. However, it is not limited thereto, and any material having flexibility and high elastic restoring force may be used to make the push member 150.

A more detailed description of the push member 150 will be described later.

The driving unit 190 transmits power received from a power providing member 170 to the push member 150.

The driving unit 190 may be combined with the push member 150 in a manner of rack-pinion gear to transmit power to the push member 150.

An embodiment of the power providing member 170 may be an electric motor that receives electric power from a battery 290 and generates power. In addition, the power providing member 170 may be various embodiments such as a manual operation handle and a hydraulic rotating means.

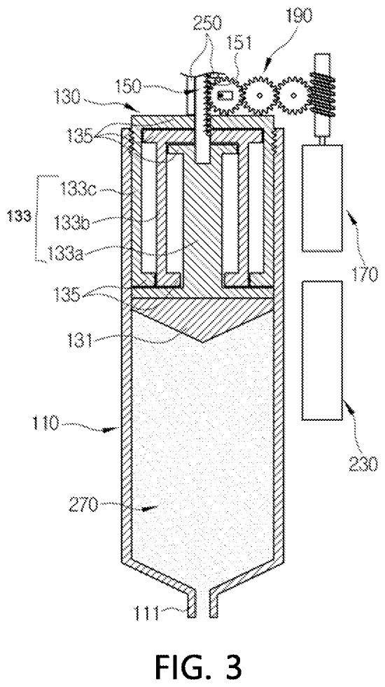

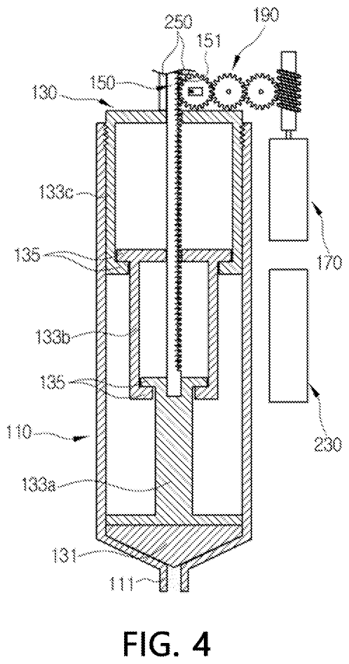

FIG. 3 is a cross-sectional view of a principal part showing a state before driving of a portable auto injection apparatus with short length according to an exemplary embodiment of the present invention and FIG. 4 is a cross-sectional view of a principal part showing a state after driving of a portable auto injection apparatus with short length according to an exemplary embodiment of the present invention.

Referring to FIGS. 3 and 4, the piston 130 may include a head 131 and a body 133, and the body 133 may include a plurality of rod members.

The head 131 contacts the injection solution 270 of the syringe 110, receives power from the push member 150 and performs a straight-line motion along the inner surface of the syringe 110, and discharges the injection solution 270 to an outlet 111.

In this case, the head 131 is in close contact with the inner surface of the syringe 110 so as not to create a gap to maintain the sealing state and performs the straight-line motion along the inner surface of the syringe 110, so the diameter of the head 131 and the inner diameter of the syringe 110 may be the same.

The body 133 provides power so that the head 131 performs straight line motion, and one end of the body 133 is connected to the head 131 and the other end is connected to one end of the syringe 110. In this case, the other end of the body may be fixed and coupled to one end of the syringe 110 so as to be detachable if necessary.

The body 133 may be configured by combining a plurality of rod members in a multi-stage structure to enable expansion and contraction, and the number of rod members may be at least two or more so as to form a multi-stage structure.

In this case, as the number of rod members increases, the length of the piston 130 may be further reduced. That is, if there are two rod members, the length of the piston 130 may be reduced to about ½ level compared to the existing one, and if there are three rod members, the length of the piston 130 may be reduced to about ⅓ level compared to the existing one.

The body 133 according to an embodiment of the present invention may include a first rod member 133a, a second rod member 133b, and a third rod member 133c in a structure of multiple stages.

The third rod member 133c may be coupled to the inner surface of the syringe 110 and may be formed in a cylindrical shape with an open inside. In addition, the third rod member 133c may have stop plates 135 with centers open at both ends thereof, respectively.

The third rod member 133c and the syringe 110 may be coupled by a screw groove coupling method or other coupling method so that the third rod member 133c does not move arbitrarily by the power transmitted from the push member 150.

The second rod member 133b may be coupled to the inner side of the third rod member 133c and may be formed in a cylindrical shape with an open inside. In addition, the second rod member 133b may have stop plates 135 with centers open at both ends thereof, respectively.

When power is transmitted from the push member 150 to the body 133, the second rod member 133b may move forward or backward along the inner wall of the third rod member 133c, and the stop plate 135 of the second rod member may be caught on the stop plate 135 of the third rod member 133b so that arbitrary movement is restricted.

The first rod member 133a may be coupled to the inner side of the second rod member 133b and may be formed in a “ [symbol] ” shape so that a stop plate 135 may be formed at one end.

When power is transmitted from the push member 150, the first rod member 133a may move forward or backward along the inner wall of the second rod member 133b, and the stop plate 135 of the first rod member 133a may be caught on the stop plate 135 of the second rod member 133b so that arbitrary movement is restricted.

In addition, openings of the stop plates 135 of the third rod member 133c and the second rod member 133b may be formed to have the same size as the diameter of the push member 150 so that the push member 150 passes through the openings and is connected to one end of the first rod member 133a.

In this case, the opening of the stop plate 135 may support the moving push member 150 from the side so that the push member 150 does not bend inside the piston 130 and the power of the driving unit 190 is delivered to the piston 130.

The stop plate 135 formed on the push member 150 described above may be formed at another end other than both ends. In addition, it may be formed in a structure of a stop groove and a stop bump instead of the stop plate 135. That is, the position of the stop plate 135 can be changed, and the stop plate 135 can be variously changed into a component that functions as the stop plate 135.

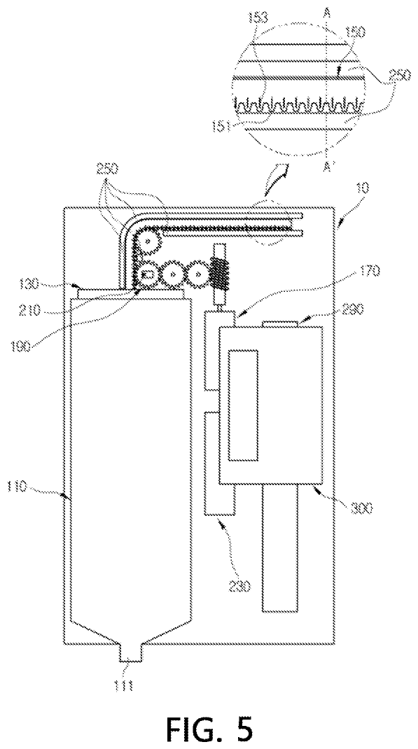

FIG. 5 is a schematic top plan view of a portable auto injection apparatus with short length according to an exemplary embodiment of the present invention.

Referring to FIGS. 2 and 5, the push member 150 may include a rack gear 151 formed on a side thereof, and the rack gear 151 may include a scale 153 formed on one side thereof.

The rack gear 151 may be coupled with the driving unit 190 to stably receive power. At this time, the rack gear 151 may be formed at a predetermined interval so that the push member 150 is precisely driven. In this case, the push member 150 may precisely control the amount of an injection solution 270 to be injected by precisely driving the piston 130.

The scale 150 is formed on one side of the rack gear 151, and a user can easily check the volume of the injected injection solution 270 through the scale 150.

In more detail, the scale 153 may be formed at the top of each tooth of the rack gear 151 formed at a predetermined interval, thereby allowing to know an exact numerical value. In this case, the portable auto injection apparatus with short length 10 may include an observation window to facilitate checking the value of the scale 153.

The portable auto injection apparatus with short length 10 may further include a sensor 210 to recognize the scale 153, a controller 230 to control the power providing member 170 by receiving information from the sensor 210, and a display 300 to output a value recognized by the sensor 210.

The sensor 210 may precisely measure the value of the scale 153. In this case, in order to precisely measure the value of the scale 153, the sensor 210 may be located above the driving unit 190 that is coupled to the rack gear 151.

The display 300 may display information from the sensor 210 to a user. In this case, the display 300 may be located on the outer surface of the portable auto injection apparatus with short length 10.

The controller 230 may control the amount of the injected injection solution 270 based on information from the sensor 210.

The portable auto injection apparatus with short length 10 may further include a support 250.

The support 250 may be formed in contact with the side of the push member 150, support the push member 150 so that it does not bend arbitrarily, and guide the push member 150 to be bent and positioned within the portable auto injection apparatus 10.

In this case, the support 250 may be formed on most side surfaces of the push member 150 except for a portion supported by the piston 130 and the driving unit 190. In addition, the support 250 may be formed in various shapes according to a position where the push member 150 is to be bent and a position where the push member 150 is to be straight.

In addition, the support 250 may be positioned close to the piston 130 to bend the push member 150. In this case, since the push member 150 guided by the support 250 is bent at a position close to the piston 130, the length of the portable auto injection apparatus with short length 10 can be shortened.

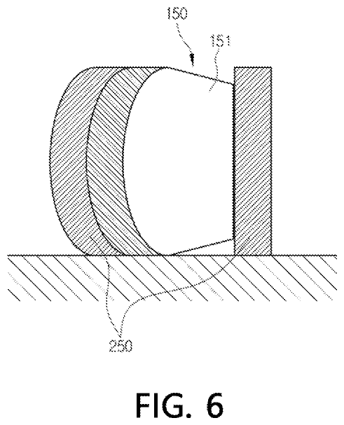

FIG. 6 is a cross-sectional view taken along line A-A′ of the portable auto injection apparatus with short length shown in FIG. 5.

Referring to FIG. 6, the push member 150 may be formed of a wire rod having a semi-curved cross-section. With this, it is possible to prevent the push member 150 from bending arbitrarily.

In addition, a rack gear 151 may be formed at predetermined intervals on a semi-curved surface. In this case, the push member 150 may be prevented once more so that it does not bend arbitrarily.

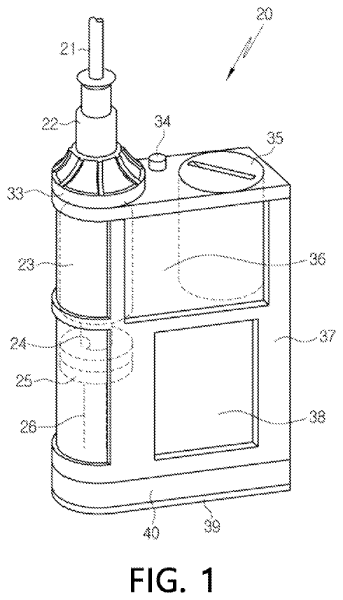

Hereinafter, the operation of the portable auto injection apparatus with short length 10 according to an embodiment of the present invention will be described with reference to FIGS. 1 to 5.

First, the piston 130 coupled with the push member 150 and the syringe 110 are coupled.

Thereafter, the push member 150 is retracted to inject the injection solution 270 into the syringe 110. In this case, the length of the piston 130 is reduced in a multistage structure.

Thereafter, the push member 150 is coupled to the driving unit 190. At this time, the push member 150 is coupled between the driving unit 190 and the support 250.

Thereafter, by operating the driving unit 190, the push member 150 transmits power to the piston 130. In this case, the injection solution 270 is discharged to the outlet 111 as the piston 130 configured in a multistage structure is expanded.

According to the present invention of the above-described configuration, the piston 130 may be formed by a plurality of rod members having a multistage structure that expands and contracts, thereby shortening the length of the piston 130.

In addition, by forming the push member 150 as a flexible push member 150 having an elastic restoring force, it is possible to shorten the length of the push member 150 while precisely injecting the injection solution 270.

With this, the portable auto injection apparatus with short length 10 is able to reduce the size by significantly shortening the length compared to the related art.

It will be apparent to those skilled in the art that the present invention is not limited to the above-described embodiments and the accompanying drawings and various substitutions, modifications and changes are possible within a range which does not depart from the technical concept of the present invention.

What is claimed is:

1. A portable auto injection apparatus with short length, comprising:
a syringe to store an injection solution;
a piston coupled to one end of the syringe and driving to expand/contract to discharge the injection solution to an outlet formed at the other end of the syringe;
a push member coupled to one end of the piston and driving the piston; and
a driving unit coupled to the push member and driving the push member.

2. The apparatus of claim 1, wherein the push member is a flexible push member having an elastic restoring force.

3. The apparatus of claim 1, wherein the piston comprises a head which is in contact with the inner surface of the syringe and moves in a sealed state and a body in which one end is coupled to the head, and the other end is fixedly coupled to one end of the syringe.

4. The apparatus of claim 3, wherein the body comprises at least two or more rod members coupled to each other in a multistage structure, and the rod member expands and contracts according to the driving of the push member.

5. The apparatus of claim 2, wherein the push member comprises a rack gear formed on a side of the push member and formed in a saw tooth shape at predetermined intervals, and the rack gear is coupled to the driving unit to drive.

6. The apparatus of claim 5, wherein the push member further comprises a scale formed on one side of the rack gear for checking an amount of the injection.

7. The apparatus of claim 6, further comprising:
a power providing member to generate power transmitted to the driving unit;
a sensor to recognize the scale; and
a controller to control the power providing member by receiving information from the sensor.

8. The apparatus of claim 7, further comprising a display to output a value recognized by the sensor.

9. The apparatus of claim 1, further comprising a support for supporting one side of the push member to guide a moving direction of the push member.

10. The apparatus of claim 1, wherein the push member is formed of a semi-curved wire rod.
